(19) United States
(12) Patent Application Publication
(10) Pub. No.: US 2011/0008400 A1
(43) Pub. Date: Jan. 13, 2011

(54) Digital Wipes And Method Of Handling

(76) Inventor: Ray Wagner, Tucson, AZ (US)

Correspondence Address: Nguyen & Tarbet, 6039 E. Grant Rd., Tucson, AZ, 85712, US

(21) Appl. No.: 12/878,833

(22) Filed: Sep. 9, 2010

Related U.S. Application Data
(63) Continuation-in-part of application No. 11/137,264, filed on May 25, 2005, now abandoned.
Application No. 11/137,264 is a continuation-in-part of application No. 11/784,720, filed on Apr. 9, 2007.
(60) Provisional application No. 60/574,515, filed on May 26, 2004.

(51) Int. Cl. A61K 8/34; A61K 8/73; A61K 8/365; A61K 8/368; A61K 8/02; A61Q 11/00

(52) U.S. Cl. 424/401; 424/49

(57) ABSTRACT

The present invention describes novel dental wipes and novel formulations for treating dental caries. The formulations may be used on the dental wipes or independently from the wipes. The wipe, when applied to the teeth, gums, tongue, and oral mucosa, helps treat dental caries and remove biofilm.

### CROSS REFERENCE

This application is a continuation-in-part and claims priority to U.S. patent application Ser. No. 11/137,264 filed May 25, 2005, U.S. patent application Ser. No. 11/784,720 filed Apr. 09, 2007, and U.S. Provisional Patent application Ser. No. 60/574,515, filed May 26, 2004, the specifications of which are incorporated herein by reference in their entirety.

### FIELD OF THE INVENTION

The present invention is directed to oral hygiene products, more particularly to a dental wipe impregnated with a dosage of plaque inhibitor such as xylitol.

### BACKGROUND OF THE INVENTION

Maintaining proper oral hygiene, for example treating dental caries on the teeth, gums, tongue, and oral mucosa, is important for adults and children or infants. It has been surprisingly discovered that a low dosage of xylitol is effective for treating dental caries. The present invention features a novel dental wipe and novel formulations for treating dental caries, for example formulations with low dosages of xylitol. The present invention also feature methods of treating dental caries on teeth. For example, the present invention describes treatment of dental caries with a dental wipe capable of depositing between about 0.4 to 1.0 g xylitol on the teeth.

### BRIEF DESCRIPTION OF THE DRAWINGS

FIG. 1 is a perspective view of a dental wipe of the present invention.

### DESCRIPTION OF PREFERRED EMBODIMENTS

### Dental Wipe

The present invention describes novel dental wipes and novel formulations for treating dental caries. The formulations may be used on the dental wipes or independently from the wipes. The wipe, when applied to the teeth, gums, tongue, and oral mucosa, helps treat dental caries and remove biofilm. As used herein, the term “teeth” may also refer to gums, tongue, and oral mucosa. For example, a method of treating dental caries on teeth also refers to a method of treating dental caries on teeth, gums, tongue, and oral mucosa.

The wipe may be constructed from a variety of non-toxic materials. For example a woven or non-woven absorbent material, for example a cellulosic or synthetic material. A wipe material includes but is not limited to a wipe constructed from non-woven polypropylene fabric and paper fibers, for example Hydroknit® (Kimberly-Clark).

The wipe may be textured (e.g., on all or a portion of the wipe), for example to provide a textured surface for enhanced removal of biofilm when rubbing the teeth, gums, tongue, and oral mucosa with the dental wipe.

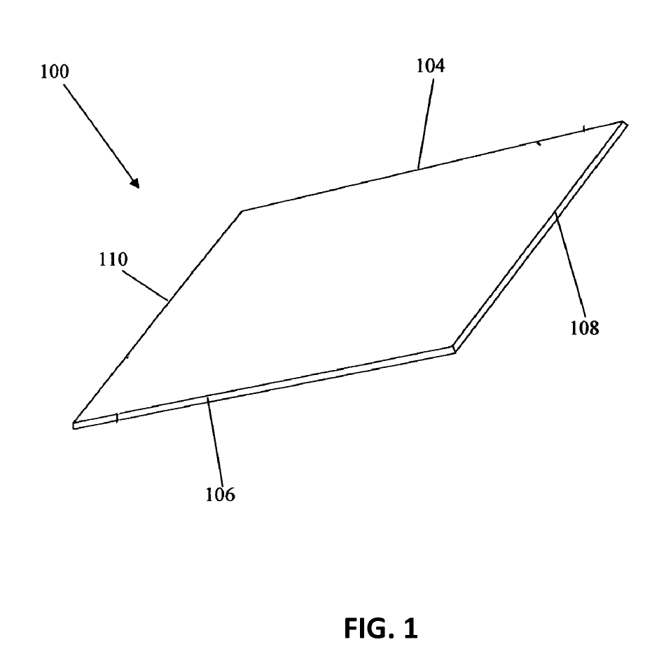

The dental wipes of the present invention may be constructed in a variety of sizes and shapes. A non-limiting example of a dental wipe 100 is shown in FIG. 1. Shapes of the dental wipes may include a rectangular shape, a circular shape, an oval shape, a triangular shape, a parallelogram shape, a trapezoid shape, a pentagon shape, a hexagon shape, an irregular shape, and/or a combination thereof. The present invention is not limited to the aforementioned shapes.

In some embodiments, the dental wipe 100 is between about 2 to 4 inches in length (e.g., as measured from a first side edge 108 to a second side edge 110). In some embodiments, the dental wipe 100 is between about 4 to 5 inches in length (e.g., as measured from a first side edge 108 to a second side edge 110). In some embodiments, the dental wipe 100 is between about 5 to 6 inches in length (e.g., as measured from a first side edge 108 to a second side edge 110). In some embodiments, the dental wipe 100 is less than about 2 inches or more than about 6 inches in length.

In some embodiments, the dental wipe 100 is between about 2 to 3 inches (e.g., 2.5 inches, 2.75 inches, etc.) in height (e.g., as measured from a third side edge 104 to a fourth side edge 106). In some embodiments, the dental wipe 100 is between about 3 to 4 inches in height (e.g., as measured from a third side edge 104 to a fourth side edge 106). In some embodiments, the dental wipe 100 is less than about 2 or more than about 4 inches in height.

The wipe is constructed to be generally durable. Generally, the wipe allows shedding of only minimal lint. However, the wipe is not limited to a material that sheds only minimal lint.

The dental wipes of the present invention are capable of depositing between about 0.4 to 1.0 g of a first plaque inhibitor onto a set of teeth when the wipe is applied to the teeth. For example, the dental wipes of the present invention are impregnated with a formulation with a dosage of a plaque inhibitor (see below).

To impregnate the dental wipe with the formulation, the dental wipe may be placed folded or unfolded into a pouch. The pouch may be subsequently filled with a volume of the solution, wherein the dental wipe absorbs all or a portion of the volume of the solution. The manufacturing process is not limited to the aforementioned example.

### Dosage and Formulation for Treating Dental Caries

The present invention also features formulation for effectively treating dental caries. The formulation comprises a dosage (e.g., low dosage) of a first plaque inhibitor, for example a range of a first plaque inhibitor between about 0.4 to 1.0 g. The first plaque inhibitor may be a sugar alcohol, for example xylitol, however the plaque inhibitor is not limited to xylitol. The formulation (e.g., dosage) may be impregnated in a dental wipe (or other material or vehicle), as described below, or may be independent of any vehicle.

The range of the dosage of first plaque inhibitor in the formulation may be between about 0.4 to 1.0 g. For example, in some embodiments, the formulation comprises a dosage between about 0.4 to 0.6 g of the first plaque inhibitor. In some embodiments, the formulation comprises a dosage between about 0.6 to 0.8 g (e.g., 0.7 g) of the first plaque inhibitor. In some embodiments, the formulation comprises a dosage between about 0.8 to 1.0 g of the first plaque inhibitor.

Dosage may be reflected alternatively with concentration. For example, in some embodiments, the formulation comprises between about 20 to 50% of the first plaque inhibitor. For example, in some embodiments, the formulation comprises between about 20 to 30% of the first plaque inhibitor (e.g., 25%). In some embodiments, the formulation comprises between about 30 to 40% of the first plaque inhibitor (e.g., 35%). In some embodiments, the formulation comprises between about 40 to 50% of the first plaque inhibitor (e.g., 45%).

The formulation has a volume, for example a volume between about 1 to 5 ml (e.g., an individual dose). For example, in some embodiments, the volume is between about 1 to 2 ml. In some embodiments, the volume is between about 2 to 3 ml. In some embodiments, the volume is between about 3 to 4 ml. In some embodiments, the volume is between about 4 to 5 ml.

The formulation may comprise one or more of the following: a carrier (e.g., including but not limited to water), a humectant (e.g., including but not limited to glycerin, an adherence component or colloidal thickening agent (e.g., including but not limited to glycerin, hydroxyethyl cellulose, a preservative (e.g. including but not limited to citric acid, trisodium citrate, sodium benzoate and a flavor component.

In some embodiments, the concentration of the adherence component is between about 0.1 to 11%. For example, in some embodiments, the concentration of the adherence component is between about 1 to 10% glycerin (e.g., between about 1 to 2% glycerin, between about 2 to 4% glycerin, between about 4 to 6% glycerin, between about 6 to 8% glycerin, between about 2 to 8% glycerin, between about 8 to 10% glycerin). In some embodiments, the concentration of adherence component is between about 0.1 to 1.0% hydroxyethyl cellulose (e.g., between about 0.1 to 0.5% hydroxyethyl cellulose, between about 0.5 to 1.0% hydroxyethyl cellulose. In some embodiments; the adherence component comprises between about 2 to 8% glycerin and between about 0.1 to 1.0% hydroxyethyl cellulose.

In some embodiments, the concentration of the preservative is between about 0.0001 to 0.06%, between about 0.0001 to 0.8%, between about 0.001 to 2.5%, etc. For example, in some embodiments, the concentration of the preservative is between about 0.002 to 1.0% citric acid (e.g., between about 0.002 to 0.02% citric acid, between about 0.02 to 0.05% citric acid, between about 0.05 to 0.1 citric acid, between about 0.1 to 0.15% citric acid, between about 0.15 to 0.2% citric acid, between about 0.02 to 0.2% citric acid, between about 0.2 to 0.5% citric acid, between about 0.5 to 1.0% citric acid). In some embodiments, the concentration of the preservative is between about 0.01 to 1.0% trisodium citrate (e.g., between about 0.01 to 0.05% trisodium citrate, between about 0.05 to 0.1% trisodium citrate, between about 0.1 to 0.2% trisodium citrate, between about 0.2 to 0.5% trisodium citrate, between about 0.5 to 1.0% trisodium citrate). In some embodiments, the concentration of the preservative is between about 0.0001 and 0.5% sodium benzoate (e.g., between about 0.0001 to 0.001% sodium benzoate, between about 0.001 to 0.01% sodium benzoate, between about 0.1 to 0.5% sodium benzoate). In some embodiments, the preservative comprises between about 0.002 to 1.0% citric acid, between about 0.01 to 1.0% trisodium citrate, and between about 0.0001 to 0.5% sodium benzoate.

In addition, in some embodiments, the formulation may further comprise an antimicrobial agent including but not limited to nystatin and/or imidazole and/or fluconazole, an antibiotic including but not limited to penicillin and/or tetracycline, an antiseptic including but not limited to chlorhexadiene, an anti-inflammatory agent including but not limited to corticosteroids (e.g., hydrocortisone), and/or an anesthetic agent including but not limited to lidocaine and/or benzocaine.

In addition, in some embodiments, the formulation may further comprise a cleansing surfactant, a dental whitening agent, a re-calcifying agent, and/or a mouth freshening agent (e.g., ethanol, menthol).

The formulation of the present invention may be impregnated into the dental wipes of the present invention. The dental wipes are capable of depositing between about 0.4 to 1.0 g of a first plaque inhibitor onto a set of teeth when the wipe is applied to the teeth.

In some embodiments, the wipe holds/absorbs between about 0.4 to 1.0 g of xylitol and can release or deposit onto the teeth between about 0.4 g to 1.0 g of xylitol. For example, the wipe may hold/absorb about 0.7 g of xylitol and can release or deposit onto the teeth about 0.5 g of xylitol (e.g., some xylitol is retained in the wipe during/after the application to the teeth). In some embodiments, the wipe holds/absorbs between about 1.0 to 2.0 g of xylitol and can release or deposit onto the teeth between about 0.4 g to 1.0 g of xylitol. In some embodiments, the wipe holds/absorbs between about 2.0 to 3.0 g of xylitol and can release or deposit onto the teeth between about 0.4 g to 1.0 g of xylitol. In some embodiments, the wipe holds/absorbs more than about 3.0 g of xylitol and can release or deposit onto the teeth between about 0.4 g to 1.0 g of xylitol.

In some embodiments, the dental wipe holds/absorbs between about 1 to 2 ml of formulation (e.g., 1.5 ml) In some embodiments, the dental wipe holds/absorbs between about 2 to 3 ml of formulation. In some embodiments, the dental wipe holds/absorbs more than about 3 ml of formulation. In some embodiments, the dental wipe holds/absorbs between about 1 to 2 ml of formulation and can release or deposit onto the teeth between about 0.5 to 1.8 ml of formulation (e.g., 1.5 ml of formulation). For example, the wipe may hold/absorb about 2 ml of formulation and can release or deposit onto the teeth about 1.5 ml of formulation. In some embodiments, the dental wipe holds/absorbs between about 2 to 3 ml of formulation and can release or deposit onto the teeth between about 1.0 to 2.8 ml of formulation (e.g., 2.5 ml of formulation).

The dental wipe likely retains a portion of the formulation and/or xylitol dosage when the dental wipe is applied to the teeth. In some embodiments, the dental wipe retains between about 0.1 to 5% of the volume of formulation. In some embodiments, the dental wipe retains between about 5 to 10% of the volume of formulation. In some embodiments, the dental wipe retains between about 10 to 20% of the volume of formulation. In some embodiments, the dental wipe retains between about 20 to 30% (e.g., 25%) of the volume of formulation. In some embodiments, the dental wipe retains between about 30 to 40% of the volume of formulation. In some embodiments, the dental wipe retains between more than about 40% of the volume of formulation.

In some embodiments, the dental wipe retains between about 0.1 to 5% of the xylitol dosage. In some embodiments, the dental wipe retains between about 5 to 10% of the xylitol dosage. In some embodiments, the dental wipe retains between about 10 to 20% of the xylitol dosage. In some embodiments, the dental wipe retains between about 20 to 30% (e.g., 25%) of the xylitol dosage. In some embodiments, the dental wipe retains between about 30 to 40% of the xylitol dosage. In some embodiments, the dental wipe retains between more than about 40% of the xylitol dosage.

### Methods of Treating Dental Caries

The present invention also features methods of treating dental caries (or methods of debridement of biofilm). It has been surprisingly discovered that a low dose of plaque inhibitor (e.g., xylitol) is sufficient for treatment of dental caries.

In some embodiments, the method comprises applying between about 1.2 g to 3 g of a first plaque inhibitor (e.g., xylitol) onto the teeth per day. In some embodiments, the method comprises applying the first plaque inhibitor (e.g., xylitol) onto the teeth in a series of individual applications (per day). For example, in some embodiments, between about 0.24 to 0.6 g of the first plaque inhibitor (e.g., xylitol) is applied to the teeth five times per day. In some embodiments, between about 0.3 to 0.75 g of the first plaque inhibitor (e.g., xylitol) is applied to the teeth four times per day. In some embodiments, between about 0.4 to 1.0 g of the first plaque inhibitor (e.g., xylitol) is applied to the teeth three times per day. In some embodiments, between about 0.6 to 1.5 g of a first plaque inhibitor (e.g., xylitol) is applied onto the teeth two times per day. In some embodiments, between about 1.2 to 3 g of the first plaque inhibitor (e.g., xylitol) is applied to the teeth once a day.

In some embodiments, the low dosage of plaque inhibitor (e.g., xylitol) is applied to the teeth via a dental wipe capable of depositing an amount of a plaque inhibitor (e.g., xylitol) onto the teeth. In some embodiments, the wipe deposits between about 0.4 to 1.0 g of the first plaque inhibitor (e.g., xylitol)

### Combination Xylitol and Erythritol

The present invention also feature a combination formulation for effectively treating dental caries comprising a dosage (e.g., a low dosage) of a first plaque inhibitor and a second plaque inhibitor. The first plaque inhibitor may be a sugar alcohol, for example xylitol, however the plaque inhibitor is not limited to xylitol. The second plaque inhibitor may be a sugar alcohol, for example erythritol.

In some embodiments, the combination formulation comprises a dosage of the first plaque inhibitor between about 0.4 to 1.0 g. For example, in some embodiments, the combination formulation comprises a dosage of the first plaque inhibitor between about 0.4 to 0.6 g of the first plaque inhibitor. In some embodiments, the combination formulation comprises a dosage of the first plaque inhibitor between about 0.6 to 0.8 g (e.g., 0.7 g) of the first plaque inhibitor. In some embodiments, the combination formulation comprises a dosage of the first plaque inhibitor between about 0.8 to 1.0 g of the first plaque inhibitor.

In some embodiments, the combination formulation comprises a dosage of the second plaque inhibitor between about 0.05 to 1.0 g. For example, in some embodiments, the combination formulation comprises a dosage of the second plaque inhibitor between about 0.05 to 0.1 g. In some embodiments, the combination formulation comprises a dosage of the second plaque inhibitor between about 0.1 to 0.5 g. In some embodiments, the combination formulation comprises a dosage of the second plaque inhibitor between about 0.5 to 1.0 g.

In some embodiments, the combination formulation comprises a dosage of the first plaque inhibitor between about 0.4 to 1.0 g and a dosage of the second plaque inhibitor between about 0.05 to 1.0 g.

In some embodiments, the combination formulation comprises between about 20 to 50% of the first plaque inhibitor. For example, in some embodiments, the combination formulation comprises between about 20 to 30% of the first plaque inhibitor. In some embodiments, the combination formulation comprises between about 30 to 40% of the first plaque inhibitor. In some embodiments, the combination formulation comprises between about 40 to 50% of the first plaque inhibitor.

In some embodiments, the combination formulation comprises between about 2.5 to 50% of the second plaque inhibitor. For example, in some embodiments, the combination formulation comprises between about 2.5 to 10% of the second plaque inhibitor. In some embodiments, the combination formulation comprises between about 10 to 25% of the second plaque inhibitor. In some embodiments, the combination formulation comprises between about 25 to 50% of the second plaque inhibitor.

In some embodiments, the combination formulation comprises between about 20 to 50% of the first plaque inhibitor and between about 2.5 to 50% of the second plaque inhibitor.

As used herein, the term “about” refers to plus or minus 10% of the referenced number. For example, an embodiment wherein the volume of formulation is about 2 ml includes a volume of formulation between 1.8 and 2.2 ml.

### Example 1

Table 1 describes ranges of components of a formulation of the present invention with a dosage of plaque inhibitor (e.g., xylitol).

TABLE 1

| Material | Amount (%) |
|---|---|
| Carrier (e.g. , water, purified water) | QS to 100% |
| 1^{st} plaque inhibitor (e.g. , xylitol) | 20 to 50% |
| Humectant | |
| 1^{st} adherence component | 0.1 to 1.0% |
| (e.g. , hydroxyethyl cellulose) | |
| 2^{nd} adherence component (e.g. , glycerin) | 1.0 to 10% |
| 1^{st} preservative (e.g. , sodium benzoate) | 0.0001 to 0.5% |
| 2^{nd} preservative (e.g. , trisodium citrate) | 0.01 to 1.0% |
| 3^{rd} preservative (e.g. , citric acid) | 0.002 to 0.1% |

### Example 2

Table 2 describes a non-limiting example of a formulation of the present invention with a dosage of plaque inhibitor (e.g., xylitol).

TABLE 2

| Material | Amount (%) |
|---|---|
| Carrier (e.g. , water, purified water) | QS to 100% |
| 1^{st} plaque inhibitor (e.g. , xylitol) | 35% |
| Humectant | |
| 1^{st} adherence component | 0.5% |
| (e.g. , hydroxyethyl cellulose) | |
| 2^{nd} adherence component (e.g. , glycerin) | 5% |
| 1^{st} preservative (e.g. , sodium benzoate) | 0.2% |
| 2^{nd} preservative (e.g. , trisodium citrate) | 0.18% |
| 3^{rd} preservative (e.g. , citric acid) | 0.02% |

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

What is claimed is:

1. A wipe for treating dental caries on teeth, said wipe is capable of depositing between about 0.4 to 1.0 g of xylitol onto the teeth when the wipe is applied to the teeth.

2. The wipe of claim 1, wherein the wipe is textured.

3. The wipe of claim 1, wherein the wipe has an absorbent capacity of between about 50 to 200% dry weight.

4. The wipe of claim 1 comprising a volume of formulation, the formulation comprising xylitol.

5. The wipe of claim 4, wherein the volume is between about 2 to 3 ml.

6. The wipe of claim 4 further comprising water.

7. The wipe of claim 4 further comprising a concentration of a humectant.

8. The wipe of claim 4 further comprising a concentration of an adherence component.

9. The wipe of claim 8, wherein the adherence component comprises glycerin, hydroxyethyl cellulose, or a combination thereof.

10. The wipe of claim 9, wherein the concentration of adherence component comprises between about 2 to 8% glycerin, between about 0.1 to 1.0% hydroxyethyl cellulose, or both between about 2 to 8% glycerin and between about 0.1 to 1.0% hydroxyethyl cellulose.

11. The wipe of claim 4 further comprising a concentration of a preservative.

12. The wipe of claim 11, wherein the preservative comprises citric acid, trisodium citrate, sodium benzoate, or a combination thereof.

13. The wipe of claim 12, wherein the concentration of the preservative comprises (i) between about 0.02 to 0.2% citric acid, (ii) between about 0.1 to 0.3% trisodium citrate, (iii) between about 0.0001 to 0.1% sodium benzoate, or (iv) between about 0.02 to 0.2% citric acid, between about 0.1 to 0.3% trisodium citrate, and between about 0.0001 to 0.1% sodium benzoate.
